(19) United States
(12) Patent Application Publication
(10) Pub. No.: US 2002/0115619 A1
(43) Pub. Date: Aug. 22, 2002

(54) Compositions and methods for treatment of cystic fibrosis

(76) Inventors: Ronald C. Rubenstein, Ardmore, PA (US); William Reenstra, Radnor, PA (US)

Correspondence Address: DANN DORFMAN HERRELL & SKILLMAN, SUITE 720, 1601 MARKET STREET, PHILADELPHIA, PA, 19103-2307, US

(21) Appl. No.: 09/970,843

(22) Filed: Oct. 4, 2001

Related U.S. Application Data
(60) Provisional application No. 60/237,899, filed on Oct. 4, 2000.

(51) Int. Cl. A61K 31/7048; A61K 31/353; A61K 31/19

(52) U.S. Cl. 514/27; 514/456; 514/557; 514/570

(57) ABSTRACT

The invention includes a method of enhancing the chloride ion transport function of a mutant CFTR polypeptide in epithelial cells in a mammal. In a preferred embodiment, the mammal is a human patient afflicted with cystic fibrosis (CF). Specifically, the method comprises administering to a patient a therapeutically effective amount of a first compound to enhance trafficking of a mutant CFTR polypeptide to the surface of epithelial cells in the patient, and a therapeutically effective amount of a second compound to increase the chloride ion transport activity of a mutant CFTR polypeptide at the surface of epithelial cells, whereby, the chloride ion transport function of the mutant CFTR polypeptide is enhanced. The invention also includes a method of treating CF in a patient, wherein a mutant CFTR polypeptide is present in an epithelial cell in a patient with CF. Compositions for treating CF in a patient are also included, as well as kits for practicing the method of the invention.

Pursuant to 35 U.S.C. §202(c) it is acknowledged that the U.S. Government has certain rights in the invention described herein, which was made in part with funds from the National Institutes of Health, USPHS Grant Number, R01 DK58046.

This application claims priority under 35 U.S.C. §119(e) to U.S. Provisional Application No. 60/237,899 filed Oct. 4, 2000.

### BACKGROUND OF THE INVENTION

Cystic Fibrosis (CF) is an autosomal recessive systemic disorder of exocrine glands and secretory epithelia. The disease is a consequence of mutations in the cystic fibrosis transmembrane conductance regulator (CFTR) gene, which cause a variety of abnormalities in CFTR protein expression and/or regulation. CFTR functions as a cAMP-regulated chloride channel in the apical membranes of epithelial cells, including: nasal, pulmonary, sweat gland, hepatic, and intestinal cells. Most of the defects in CF result from reduced chloride ion transport. Recent improvements in CF diagnosis and the treatment of lung disease have improved the median survival for patients with this disorder to greater than 30 years, but respiratory failure from chronic infections remains the most common cause of death in CF.

More than one thousand unique disease causing mutations have been identified in the CFTR gene. These mutations can be classified in five general categories with respect to the CFTR protein (Table 1). These classes of CFTR dysfunction include limitations in CFTR production (Class I), aberrant folding and/or trafficking (Class II), abnormal regulation of conduction (Class III), decreases in chloride conduction (Class IV), and reductions in synthesis (Class V). Due to the lack of functional CFTR, Class I, II, and III mutations are typically associated with a more severe phenotype in CF (i.e. pancreatic insufficiency) than the Class IV or V mutations, which may have very low levels of functional CFTR expression.

The most common mutation, ΔF508, is present on over 60% of CF chromosomes and greater than 85% of all CF patients have at least one ΔF508-CFTR gene. ΔF508 is considered the prototype Class II trafficking mutation. ΔF508 encodes a cAMP-activated chloride channel with reduced activity in cells (Dalemans et al., 1991, Nature 354:526-528; Drum et al., 1991, Science 254:1797-9; Hwang et al., 1997, Am. J. Physiol. 273 (Cell Physiol. 42):C988-C998) which is also misprocessed in the endoplasmic reticulum (Cheng et al., 1990, Cell 63: 827-834; Ward et al., 1994, J. Biol. Chem. 269:25710-25718). The absence of cell surface CFTR caused by this trafficking defect is typically associated with a severe phenotype of CF, including pancreatic insufficiency. When expressed in systems which facilitate protein trafficking studies such as Xenopus oocytes (Drumm et al., 1991, Science 254: 1797-1799), or in high level expression systems that allow some ΔF508-CFTR to reach the cell surface (Cheng et al., 1995, Am. J. Physiol. 268: L615-L624), ΔF508-CFTR is found to be less active than wild type CFTR. Several experimental conditions, however, have been shown to increase the activity of ΔF508-CFTR to levels that approach or exceed those of wild type CFTR (Drumm et al., 1991, Science 254:1797-9; Hwang et al., 1997, Am. J. Physiol. 273 {Cell Physiol. 42}:C988-C998).

TABLE 1
Functional Classification of CFTR Mutations

| | | Class II | | | |
|---|---|---|---|---|---|
| DNA | Class I | Missense or | Class III | Class IV | Class V |
| mutation | Nonsense | Deletion | Missense | Missense | Intron |
| mRNA | ↓↓ | + | + | + | ↓ |
| Protein | − | + | + | + | ↓ |
| Synthesis | | | | | |
| Intracellular | − | ↓↓ | + | + | ↓ |
| Trafficking | | | | | |
| or processing | | | | | |
| Function | − | − | − | ↓ | ↓ |

+ Present
− Absent
↓ Reduced
↓↓ Greatly reduced

Recently, a number of groups have begun investigating a novel therapeutic approach, coined “protein-repair therapy.” This approach, when directed to the study of cystic fibrosis, aims to understand the molecular defects associated with mutant CFTR polypeptides, and to direct pharmacological therapy to correct the CFTR dysfunction in a mutation-specific fashion. Based on its preserved function but abnormal intracellular trafficking, a number of groups have investigated protein repair strategies to improve intracellular trafficking of ΔF508-CFTR. Successful approaches have included incubating cells expressing ΔF508-CFTR at reduced temperature (Denning et al., 1992, Nature 358: 761-764), in the presence of high concentrations of protein stabilizing agents such as glycerol (Brown et al., 1996, Cell Stress and Chaperones 1:117-125; Sato et al., 1996, J. Biol. Chem. 271: 635-638) or in the presence of the transcriptional regulator butyrate (Cheng et al., 1995, Am. J. Physiol. 268: L615-L624). It has also been demonstrated that an orally bioavailable analog of butyrate, 4-phenylbutyrate (4PBA), can also improve the aberrant intracellular trafficking of the ΔF508-CFTR protein and lead to some degree of CFTR function on the cell surface of CF epithelial cells in vitro (Rubenstein et al., 1997, J. Clin. Invest. 100: 2457-2465).

Since 4PBA is an FDA approved pharmaceutical for use in patients with urea cycle disorders, a pilot clinical trial of 4PBA was carried out in CF subjects homozygous for the ΔF508-CFTR mutation (Rubenstein and Zeitlin, 1998, Am. J. Resp. Crit.Care Med. 157: 484-490). In this randomized, placebo controlled, double blind study, subjects received either 4PBA or placebo at a dose of 19 grams per day divided t.i.d. (the standard adult dose of 4PBA is 20 g/day). After one week of study drug therapy, a small but statistically significant improvement in the Nasal Potential Difference measurements (NPD) of subjects who had received 4PBA, but not in subjects who had received placebo, was observed. The improved NPD measurements of the 4PBA-treated subjects, however, was more like NPD measurements of subjects with CF than NPD measurements of non-CF subjects. Thus, these data are consistent with 4PBA improving CFTR function in ΔF508-CFTR-homozygous CF subjects, but not to non-CF levels. Importantly, there were no significant side effects reported during this trial that were related to 4PBA therapy.

Several compounds have been shown to increase the cAMP-dependent activity of ΔF508-CFTR, they include isobutylmethyl xanthine (Drumm et al., 1991, Science 254:1797-9) 8-cyclopentyl-1,3-dipropylxanthine (CPX) (Eidelman et al., 1992, Proc. Natl. Acad. Sci U.S.A. 89:5562-5566), and genistein (Hwang et al., 1997, Am. J. Physiol. 273 (Cell Physiol. 42:C988-C998; He et al., 1998, Am. J. Physiol. Cell Physiol. 275:C958-C966). Of these compounds, genistein is the most interesting, as it does not activate ΔF508-CFTR by itself but rather enhances cAMP-dependent activation by as much as 20-fold (Hwang et al., Am. J. Physiol. 273 {Cell Physiol. 42}:C988-C998).

Genistein is a component of soy products which is absorbed orally. It has been linked to reduced rates of cancer in both humans and rodents and is currently being tested for its ability to inhibit prostate cancer (Gray et al., 1979, Brit. J. Can. 39:1-7; Severson et al., 1989, Can. Res. 49:1857-1860; and Lamartiniere et al., 1995, Carcinogenesis. 16:2833-2840). In contrast to genistein, the concentration of isobutylmethyl xanthine needed to increase ΔF508-CFTR activity is inconsistent with its use in human subjects (Drumm et al., 1991, Science 254:1797-9).

### SUMMARY OF THE INVENTION

The present invention addresses the need for improved therapeutic approaches for the treatment of CF patients. In accordance with the one aspect of the present invention, a treatment method is provided in which CF patients are treated with a combination of therapeutic agents. Such combination therapy serves to augment the beneficial effects of individual therapeutic agents, thereby providing a more efficacious clinical protocol for the treatment of CF patients. The present invention also provides methodology with which to screen for additional pharmaceutical agents that can augment the therapeutic benefits of 4PBA therapy for CF patients.

According to another aspect of the present invention, a therapeutic regimen is provided for the treatment of a mammal having a mutated CFTR. The therapeutic regimen includes a method for enhancing the chloride ion transport function of a mutant CFTR polypeptide in an epithelial cell in a mammal. The method comprises a) administering to a mammal a therapeutically effective amount of a first compound to enhance the trafficking of a mutant CFTR polypeptide to the surface of an epithelial cell; and b) administering to a mammal a therapeutically effective amount of a second compound to increase the chloride ion transport activity of the mutant CFTR polypeptide, thereby enhancing the function of the mutant CFTR polypeptide.

In one embodiment of the present invention, the epithelial cell is present in a mammal afflicted with CF.

In a preferred embodiment, the mammal afflicted with CF is a human. According to one aspect of the present invention, methods are provided for enhancing the activity of mutant CFTR polypeptides in epithelial cells in non-pediatric CF patients with combination therapy.

According to a further aspect of the present invention, methods are provided for enhancing the activity of mutant CFTR polypeptides in epithelial cells in pediatric CF patients, wherein the therapy has been optimized for such patients.

According to one aspect of the present invention, the mutant CFTR polypeptide is a Class II mutant, which is defective in CFTR trafficking.

In a preferred embodiment, the mutant CFTR polypeptide is N1303K, ΔI507, A455E, R347P, S549R, S549I, and A559T.

In a particularly preferred embodiment, the mutant CFTR polypeptide is ΔF508-CFTR.

In another aspect of the present invention, the epithelial cell is a nasal epithelial cell, a lung epithelial cell, a pancreatic epithelial cell, an intestinal epithelial cell, a biliary epithelial cell and/or a sweat duct epithelial cell.

In one embodiment, the first compound is butyrate, phenylbutyrate, 4-phenylbutyrate, or a biologically active analog of butyrate or phenyl butyrate. In another embodiment, the second compound is an isoflavone or a flavone.

In a particularly preferred embodiment, the second compound is genistein or a biologically active analog thereof.

In one aspect, the first compound and the second compound are administered to the mammal together as components of the same composition.

In yet another aspect, the first compound and the second compound are administered to a mammal as components of different compositions.

In one embodiment, the first compound is administered to a mammal prior to administering the second compound.

In another embodiment, the first compound is administered to the mammal from about 4 hours to about 48 hours prior to administering the second compound to the mammal.

In one aspect, the first compound is administered to the mammal systemically.

In another aspect, the first compound is administered to the mammal topically.

In another aspect, the second compound is administered to the mammal topically, parenterally, orally, intravenously and/or by inhalation.

In accordance with the present invention, it has been discovered that a percentage of CF patients develop tolerance to therapeutic compounds used for the treatment of the disorder. Thus, in a particularly preferred embodiment of the present invention, the combination of therapeutic agents comprising a first compound and a second compound is administered to a non-pediatric or pediatric patient having a mutated CFTR following a chronic intermittent schedule. Such a schedule can be utilized to avoid the development of tolerance to one or both of the first and second compounds of the present invention.

A preferred schedule for chronic intermittent treatment provides for one to two weeks of administration of a first and a second compound of the present invention followed by a two to four week period in which a patient is not treated with the first and second compound. In methods wherein 4-phenylbutyrate (4PBA) is the first compound, it is administered to an adult (non pediatric) patient systemically in a dosage range of 15 to 30 grams per day. In a preferred embodiment, 4PBA is administered to an adult patient systemically in a dosage range of 20 to 27 grams per day. In methods wherein 4PBA is the first compound, it is administered to a pediatric patient systemically (i.e., less than about 40 kilograms in weight) in a dosage range from about 100 to about 600 milligrams per kilogram per day. In a preferred embodiment, 4PBA is administered to a pediatric patient systemically in a dosage range of 300 to about 500 milligrams per kilogram per day. In methods wherein genistein is the second compound, it is administered in a dosage range of about 10 to about 30 milligrams per kilogram per day, and is preferably about 16 milligrams per kilogram per day. In another embodiment, 4PBA and genistein can be administered to a patient following a schedule for chronic intermittent treatment wherein the genistein is administered two to four days after 4PBA.

A preferred schedule of chronic intermittent treatment involves administration of a second compound of the present invention in conjunction with a first compound, wherein the second compound may be delivered concurrently with or after administration of the first compound. In a particularly preferred embodiment, a second compound is administered when a patient has not developed tolerance to a first compound of the present invention.

This combined therapeutic approach may extend the duration of amelioration of CF disease symptoms and may also provide a safe and effective long term regimen for treatment of CF patients. More specifically, the method of the present invention is expected to improve the short and long term prognosis of patients afflicted with CF.

The invention also includes a kit for treating cystic fibrosis in a human patient. The kit comprises a) a first compound in a therapeutically effective amount to enhance the trafficking of a mutant CFTR polypeptide to the surface of an epithelial cell in a human patient; b) a second compound in a therapeutically effective amount to increase the chloride ion transport activity of a mutant CFTR polypeptide; and c) an instructional material which may optionally include a chronic intermittent dosing schedule for administration of compound 1 and/or 2 to ensure that a state of tolerance is not induced and also directs the use of a) and b) for the function of treating cystic fibrosis in a human patient.

In another aspect, the kit optionally comprises a device for providing delivery of one or more of the first compound and the second compound in an aerosolized formulation.

### BRIEF DESCRIPTION OF THE DRAWINGS

The foregoing summary, as well as the following detailed description of the invention, will be better understood when read in conjunction with the appended drawings.

FIG. 1 is a graph depicting typical results of nasal potential difference measurements in patients with CF and in non-CF patients.

FIG. 2 is a graph depicting typical results of nasal potential difference measurements in patients homozygous for the ΔF508-CFTR mutation after therapy with 4-phenyl butyrate (4PBA).

FIG. 3 is a graph depicting the results of nasal potential difference measurements for a patient with CF who is homozygous for the ΔF508-CFTR mutation and was treated in a blinded clinical trial to determine the combined effects of administration of 4PBA and genistein. The patient demonstrated a response to blinded study drug therapy that was consistent with that observed in patients who had received 4PBA in previous clinical trials (depicted in FIG. 2). The patient had a 2/3 chance of receiving 4PBA and a 100% chance of receiving genistein in this study. (See Example 1 below for a more complete description of this trial)

FIG. 4 shows current/voltage (I/V) curves determined in oocytes injected with either A) ΔF508-CFTR (10 ng) cRNA prior to (●) and after (∘) stimulation with 10 μM forskolin/100 μM IBMX (n=19) or B) rat αβγENac (0.33 ng/subunit) cRNAs prior to (▪) and after (□) addition of 10 μM amiloride (n=9). Data are expressed as the mean ±S.E.M.

FIG. 5 shows measurements of whole-cell currents obtained from oocytes injected wild type CFTR (WT-CFTR; 10 ng) cRNA, before (white bar) and after (black bar) addition of 10 μM forskolin/100 μM IBMX (n=9). Whole-cell amiloride-inhibited currents (10 μM amiloride) measured in oocytes injected with αβγENac (0.33 ng/subunit) cRNAs, before (gray bar) and after (dashed black bar) stimulation with 10 μM forskolin/100 μM IBMX (n=10). Oocytes were injected with WT-CFTR (10 ng) and αβγENac (0.33 ng/subunit) cRNAs. Whole cell amiloride-sensitive currents were measured before (gray bar) and after (dashed black bar) stimulation with 10 μM forskolin/100 μM IBMX. The black bars represent the whole cell current measured after addition of 10 μM forskolin/100 μM IBMX which was amiloride-insensitive and was therefore reflective of WT-CFTR mediated current (n=6). The whole cell currents were determined at a holding potential of −100 mV. Data are expressed as the mean ±S.E.M.

FIG. 6 shows measurements of whole-cell currents obtained from oocytes injected with ΔF508-CFTR (10 ng) cRNA, before (white bar) and after (black bar) addition of 10 μM forskolin/100 μM IBMX (n=19). Whole-cell amiloride-inhibited currents (10 μM amiloride) measured in oocytes injected with αβγENac (0.33 ng/subunit) cRNAs, before (gray bar) and after (dashed black bar) stimulation with 10 μM forskolin/100 μM IBMX, (n=10). Oocytes were injected with ΔF508-CFTR (10 ng) and αβγENac (0.33 ng/subunit) cRNAs. Whole cell amiloride-sensitive currents were measured before (gray bar) and after (dashed black bar) stimulation with 10 μM forskolin/100 μM IBMX. The black bars represent the whole cell current measured after addition of 10 μM forskolin/100 μM IBMX which was amiloride-insensitive and was therefore reflective of ΔF508-CFTR mediated current (n=19). The whole cell currents were determined at a holding potential of −100 mV. Data are expressed as the mean ±S.E.M.

FIG. 7 shows the effect of genistein on the functional regulation of EnaC and either ΔF508 or WT CFTR. A)I/V curve determined in oocytes injected with ΔF508-CFTR (10 ng) cRNA prior to (●) and after stimulation with 10 μM forskolin/100 μM IBMX/50 μM genistein (∘) (n=24). B) I/V curve determined in oocytes injected with WT-CFTR (10 ng) cRNA prior to (▴) and after (Δ) stimulation with 10 μM forskolin/100 μM IBMX/50 μM genistein (n=14). C) I/V curve determined in oocytes injected with rat αβγENac (0.33 ng/subunit) cRNAs stimulated by 10 μM forskolin/100 μM IBMX/50 μM genistein prior to (□) and after (▪) addition of 10 μM amiloride (n=23). Data are expressed as the mean ±S.E.M.

FIG. 8 demonstrates that wild type CFTR/ENaC regulatory controls are restored when ΔF508-CFTR and ENaC are treated with genistein. Whole-cell currents obtained from oocytes injected with ΔF508-CFTR (10 ng) cRNA before (white bars) and after (black bars) addition of 10 μM forskolin/100 μM IBMX and after addition of 50 μM genistein (stippled black bars) (n=23). Whole-cell amiloride-inhibited currents (10 μM amiloride) measured in oocytes injected with αβγENaC (0.33 ng/subunit) cRNAs, before (gray bars) and after (dashed black bars) (n=23) stimulation with 10 μM forskolin/100 μM IBMX/50 μM genistein. Oocytes were injected with ΔF508-CFTR (10 ng) and αβγENaC (0.33 ng/subunit) cRNAs. Whole cell amiloride-sensitive currents were measured before (gray bars) and after (dashed black bars) stimulation with 10 μM forskolin/100 μM IBMX/50 μM genistein. The black stippled bars represent the whole cell current measured after addition of 10 μM forskolin/100 μM IBMX/50 μM genistein that was amiloride-insensitive and was therefore reflective of ΔF508-CFTR mediated current (n=19). The whole cell currents were determined at a holding potential of −100 mV. Data are expressed as the mean ±S.E.M.

FIG. 9 demonstrates the effects of genistein on normal regulatory controls wild type CFTR and EnaC. See FIG. 8 for experimental specifics.

FIG. 10 shows the whole cell current determined in oocytes injected with ΔF508-CFTR (10 ng) alone (white bars) or co-injected with αβγENaC (0.33 ng/subunit; black bars). The relative ΔF508-CFTR-mediated current was determined after stimulation with 10 μM forskolin/100 μM IBMX/50 μM genistein in ND 96 bath solution (NaCl buffer) or in N-Methyl-D-Glucamine (NMDG)-Cl bath solution (no sodium). The whole cell currents were determined at a holding potential of −100 mV. Data are expressed as the mean ±S.E.M.

### DETAILED DESCRIPTION OF THE INVENTION

The present invention relates to improved methods for the treatment of CF in a mammal. The invention includes a combination therapy method for enhancing the trafficking and chloride ion transport activity of a mutant CFTR polypeptide in an epithelial cell in a mammal. The improvement in the trafficking and chloride ion transport activity of the mutant CFTR polypeptide in an epithelial cell in a mammal afflicted with CF results in improved chloride ion transport function of the mutant CFTR polypeptide, thereby treating CF in the mammal.

The invention also includes a composition for the treatment of CF in a mammal. The composition comprises a first compound in a therapeutically effective amount to enhance the trafficking of the mutant CFTR polypeptide to the surface of an epithelial cell in the mammal, and a second compound in a therapeutically effective amount to increase the chloride ion transport activity of the mutant CFTR polypeptide in the epithelial cell in a mammal. The composition of the invention can be in the form of a pharmaceutical composition. Also included in the compositions of the invention is a kit for performing the method of the invention.

### Definitions

As used herein, each of the following terms has the meaning associated with it in this section. The articles “a” and “an” are used herein to refer to one or to more than one (i.e., to at least one) of the grammatical object of the article. By way of example, “an element” means one element or more than one element.

According to the present invention, a pharmaceutically useful agent or compound which is given to an individual is preferably administered in a “prophylactically effective amount” or a “therapeutically effective amount” (as the case may be, although prophylaxis may be considered therapy), this being sufficient to show benefit to the individual.

As used herein, a “biologically active analog” in the context of the first compound or the second compound discussed in the methods and compositions of the invention means a compound which, with regard to the first compound, has a chemical structure which is different from the first compound, but which retains the functional property of being capable, when present in an effective amount, of enhancing the trafficking of a mutant CFTR polypeptide to the surface of an epithelial cell in a mammal. A “biologically active analog” of the second compound is a compound which has a chemical structure which is different from the second compound, but which retains the functional property of being capable, when present in a therapeutically effective amount, of increasing the chloride ion transport activity of a mutant CFTR polypeptide at the surface of an epithelial cell in a mammal.

As used herein, an “instructional material” includes a publication, a recording, a diagram, or any other medium of expression which directs or dictates the use of the components of a kit for performing the function of a method of the invention described herein. The instructional material of the kit of the present invention may, for example, be affixed to a container which contains the composition or be shipped together with a container which contains the composition. Alternatively, the instructional material may be shipped separately from the container with the intention that the instructional material and the composition be used cooperatively by the recipient.

As used herein, “treating cystic fibrosis” or “to treat cystic fibrosis” in a mammal means one or more of ameliorating a symptom or symptoms of, correcting an underlying molecular or physiological disorder of, or reducing the frequency or severity of a pathological or deleterious physiological consequence of cystic fibrosis in the mammal. By way of example, and not by limitation, such symptoms, molecular or physiological disorders and deleterious physiological consequences of cystic fibrosis include pancreatic insufficiency, chronic lung disease, malnutrition, malabsorption, nasal polyps, male infertility, growth failure and shortened life expectancy.

As used herein, “to increase the chloride ion transport activity” of a mutant CFTR polypeptide in an epithelial cell in a mammal means to provide a statistically significant increase in the level of chloride ion transport activity of the mutant CFTR polypeptide in the epithelial cell in the mammal relative to the level of the chloride ion transport activity of a mutant CFTR polypeptide in an otherwise identical epithelial cell of a mammal which is not subjected to the method or composition of the invention. The level of chloride ion transport activity of a mutant CFTR polypeptide can be assessed by a skilled artisan using any method utilized for assessing the chloride ion transport activity of a polypeptide. Such methods include measurements of Nasal Potential Difference (NPD), of sweat test by pilocarpine iontophoresis, and of adrenergic stimulated sweat rate (Callen et al., 2000, J Pediatr. 137:849-55. The ordinarily skilled artisan will be aware of factors which affect whether an increase in chloride ion transport activity is to be considered statistically significant, in view of factors such as the age, gender and weight of the mammal, the severity of the cystic fibrosis in the patient, etc. The statistical significance of the increase can be determined using any mathematical or statistical method known to the skilled artisan.

As used herein, “to enhance the trafficking of a mutant CFTR polypeptide to the surface of an epithelial cell in a mammal” means to provide a statistically significant increase in the level of transport or expression of a mutant CFTR polypeptide to, at, or near the surface of an epithelial cell in the mammal, relative to the level of trafficking of the mutant CFTR polypeptide to, at, or near the surface of an otherwise identical epithelial cell in a mammal which is not subjected to the method or composition of the invention. The level of trafficking of the mutant CFTR polypeptide to, at, or near the surface of the epithelial cell can be assessed by any method known to a skilled artisan for assessing the trafficking of a polypeptide to, at, or near the epithelial cell surface. The ordinarily skilled artisan will be aware of factors which affect whether an increase in trafficking is to be considered statistically significant in view of factors such as the age, gender and weight of the mammal, the severity of the cystic fibrosis in the patient, etc. The statistical significance of the increase can be determined using any mathematical or statistical method known to the skilled artisan.

As used herein, the term “chronic intermittent treatment” refers to repeated treatment with a compound of a duration wherein the benefit of the treatment is maintained/maximized throughout the duration of the treatment, and treatments are separated by periods of sufficient duration such that repeated treatment does not lessen the benefit of the treatment.

As used herein, the term “chronic intermittent treatment schedule” refers to the prescribed times and mehtods by which a compound or combination of compounds can be given to maximize benefit and avoid tolerance. An exemplary chronic intermittent treatment schedule of administration of the compounds of the invention entails the administration of compound 1 for one-two weeks in conjunction with the administration of compound 2. This is followed by a two-four week washout schedule in which the patient is given neither compound. Following the washout period, the treatment schedule is resumed.

As used herein, the term “drug tolerance” refers to a loss of or failure to respond physiologically in a manner typically caused by drug therapy/use.

As used herein, the term “functional synergism” refers to the observation of a physiological effect of a combination of agents that supersedes the expected effect of the agents given alone.

### Description

The invention includes a method of enhancing the chloride ion transport function of a mutant CFTR polypeptide in an epithelial cell in a mammal. Specifically, the method comprises administering to a mammal an amount of a first compound effective to enhance the trafficking of the mutant CFTR polypeptide to the surface of the epithelial cell, and an amount of a second compound effective to increase the chloride ion transport activity of the mutant CFTR polypeptide, whereby the chloride ion transport function of the mutant CFTR polypeptide is enhanced. Thus, the method is useful for treating CF in a mammal afflicted with CF, wherein the mutant CFTR polypeptide is expressed in epithelial cells of the mammal.

In preferred embodiments of the invention, the mammal is a human that has CF and thus the method is used to treat a patient with CF.

In other preferred embodiments of the invention, the mutant CFTR polypeptide is ΔF508-CFTR. The invention should not, however, be construed to be limited solely to the use of this mutant form of CFTR. Rather, the invention should be construed to include other mutant forms of CFTR having similar characteristics, including, but not limited to ΔI507, S549R, S549I, A559T and N1303K.

The epithelial cell in which trafficking of the mutant CFTR polypeptide is affected is preferably a nasal epithelial cell, a lung epithelial cell, a pancreatic epithelial cell, an intestinal epithelial cell, a biliary epithelial cell, and/or a sweat duct epithelial cell.

With respect to the first compound used in the method of the invention, and also as a component of the composition of the invention described elsewhere herein, the first compound can be any compound which is capable of enhancing the trafficking of the mutant CFTR polypeptide. The enhancement in the trafficking of the mutant CFTR polypeptide can be brought about by any mechanism known to a skilled artisan. By way of example and not by limitation, the enhancement can be accomplished by activating the transcription of CFTR, by alterations in molecular chaperone expression and interaction with mutant CFTR, and by the stabilization of mutant CFTR.

The first compound is preferably butyrate, phenylbutyrate, 4-phenylbutyrate, and/or a biologically active analog of butyrate or phenyl butyrate.

With respect to the second compound used in the method of the invention, and also as a component of the composition of the invention described elsewhere herein, the second compound can be any compound capable of increasing the chloride ion transport activity of the mutant CFTR polypeptide. The enhancement in the chloride ion transport activity of the mutant CFTR polypeptide can be brought about by any mechanism known a skilled artisan. By way of example and not by limitation, the enhancement can be accomplished by increasing the probability of the CFTR ion channel being in the open state.

The second compound is preferably an isoflavone and/or a flavone. Even more preferably, the second compound is genistein, or a biologically active analog thereof.

It is not necessary that the first and second compounds be components of the same composition, in that they may be prepared as two separate compositions, although they may also be components of the same composition. Further, the first and the second compound may be administered simultaneously to the mammal, or they may be administered at different times relative to each other. For example, the first compound may be administered to the mammal prior to administering the second compound. In one embodiment, the first compound is administered to the mammal at any time from about 4 to about 48 hours prior to administering to the mammal the second compound. Preferably, the first compound is administered to the mammal from about 4 hours to about 12 hours prior to administering to the mammal the second compound. In another embodiment, the first compound is administered to the mammal at any time from about 48 hours prior to about 14 days after administering to the mammal the second compound.

The exact manner in which the first and second compounds may be administered to a patient can be determined by a skilled clinician and may vary depending on a number of factors, including, but not limited to, the age and gender of the patient, the class of CFTR mutation or mutations underlying the disease, the severity of the disease, the medical history of the patient in general and with specific regard to previous and/or ongoing drug treatment. The precise manner for administration of the first and second compounds to the patient may therefore be readily determined by a skilled clinician in view of the above criteria and other medical guidelines generally considered when developing a strategy for therapeutic intervention.

The first compound is administered to the mammal in a therapeutically effective amount to enhance the trafficking of a mutant CFTR polypeptide to the surface of the epithelial cell in a mammal, and the second compound is administered to the mammal in a therapeutically effective amount to increase the chloride ion transport activity of the mutant CFTR polypeptide. The dosage of first and second compounds to be administered to the patient may vary, however, depending on the criteria described above. Thus, it is anticipated that a dosage of the first compound for pediatric patients (i.e., less than about 40 kilograms in weight) may vary from about 100 to about 600 milligrams per kilogram per day, and is preferably from about 300 to about 500 milligrams per kilogram per day. For adults, it is anticipated that a systemically delivered dosage of the first compound may vary from about 15 to about 30 grams per day, and is preferably from about 20 to about 30 grams per day. With regard to the second compound, it is anticipated that a dosage may vary from about 10 to about 30 milligrams per kilogram per day, and is preferably about 16 milligrams per kilogram per day.

The first and second compounds can be administered to a human by any route of administration known to a skilled clinician. The route of administration of the first and second compounds to a human may, however, vary depending on the criteria as described above.

Preferably, the first compound is administered to the patient systemically, and the second compound is administered to the patient either topically or systemically. Non-limiting examples of administration systemically include administration orally, parenterally, by inhalation, by using an aerosol and intravenously. Inhalation can be utilized as means for topical delivery, depending on alveolar versus airway deposition of a compound. Such differential deposition is largely a function of the absorptive properties of a drug across alveolar epithelia.

The level of chloride ion transport activity of a mutant CFTR polypeptide in an epithelial cell in a patient can be assessed using methods which are known to a clinician trained in the treatment or diagnosis of CF. Measurement of nasal potential difference (NPD), as described in detail in the Experimental Examples herein, is an example of such a method. An example of another method for assessing chloride ion transport activity in a patient is the sweat gland chloride transport test which is described in U.S. Pat. No. 5,976,499, which is incorporated herein by reference.

Methods for assessing the level of trafficking of a mutant CFTR polypeptide to the surface of an epithelial cell in a patient are also known to a skilled artisan. Such methods include, by way of example and not by limitation, immunoprecipitation methods, phosphorylation assays utilizing protein kinases, immunoblotting, and the measurement of chloride efflux (See, for example, Rubenstein et al., 1990, Cell, 63:827; and 1997, J. Clin. Invest., 100:2457-2465).

By administering to the patient the first and second compounds as discussed above, the chloride ion transport function of the mutant CFTR polypeptide in epithelial cells of the patient is enhanced.

There is also provided in the invention a method of treating cystic fibrosis in a mammal, preferably a human. The method comprises administering to the human patient a first compound in a therapeutically effective amount to enhance the trafficking of the mutant CFTR polypeptide to the surface of an epithelial cell in the patient. The first compound is the same as the first compound described hereinabove.

The method also comprises administering to a patient a second compound in a therapeutically effective amount to increase the chloride ion transport activity of the mutant CFTR polypeptide at the surface of the epithelial cell. The second compound can be any compound capable of increasing the chloride ion transport activity of the mutant CFTR polypeptide. The enhancement in the chloride ion transport activity of the mutant CFTR polypeptide can be facilitated by a number of means, including, but not limited to, increasing the probability of the CFTR ion channel being in the open state, increasing the number of CFTR ion channels at the plasma membrane by increasing transcription and/or increasing transport of the CFTR to the membrane, stabilizing expression of CFTR at the plasma membrane by increasing the half-life of the protein and/or decreasing its rate of internalization, and by increasing its ability to regulate other epithelial ion transporters.

As a result of administering the first and second compounds as described above, the chloride ion transport function of the mutant CFTR polypeptide is enhanced at the surface of epithelial cells, thereby treating cystic fibrosis in a patient.

As discussed elsewhere herein, it is not necessary that the first and second compounds be components of the same composition, in that they may be prepared as two separate compositions, although they may also be components of the same composition. Moreover, the first and second compound can be administered to a patient using any of the routes or methods of administration discussed herein.

In a preferred embodiment, the mutant CFTR polypeptide is ΔF508-CFTR. The invention, however, should not be construed to be limited solely to the treatment of CF patients having this particular mutant form of CFTR. Rather, the invention should be construed to include other mutant forms of CFTR with similar characteristics, including, but not limited to ΔI507, S549R, S549I, A559T and N1303K.

Also in a preferred embodiment, the epithelial cell is a nasal epithelial cell, a lung epithelial cell, a pancreatic epithelial cell, an intestinal epithelial cell, a biliary epithelial cell, and/or a sweat duct epithelial cell.

In a particularly preferred embodiment, methods are provided for treating cystic fibrosis in a non-pediatric or pediatric patient, wherein a chronic intermittent schedule is followed for the administration of a combination of therapeutic agents comprising a first compound and a second compound of the present invention. Such a schedule is designed to avoid the development of a state of tolerance to one or both of the first and second compounds of the present invention.

A preferred schedule for chronic intermittent treatment provides for one to two weeks of administration of a first and a second compound of the present invention followed by a two to four week period in which a patient is not treated with the first and second compound. In methods wherein 4-phenylbutyrate (4PBA) is the first compound, it is administered to an adult (non pediatric) patient systemically in a dosage range of 15 to 30 grams per day. In a preferred embodiment, 4PBA is administered to an adult patient systemically in a dosage range of 20 to 27 grams per day. In methods wherein 4PBA is the first compound, it is administered to a pediatric patient(i.e., less than about 40 kilograms in weight) systemically in a dosage range of about 100 to 600 milligrams per kilogram per day. In a preferred embodiment, 4PBA is administered to a pediatric patient systemically in a dosage range of about 300 to 500 milligrams per kilogram per day. In methods wherein genistein is the second compound, it is administered in a dosage range of about 10 to about 30 milligrams per kilogram per day, and is preferably about 16 milligrams per kilogram per day. In another embodiment, 4PBA and genistein can be administered to a patient following a schedule for chronic intermittent treatment wherein the genistein is administered two to four days after 4PBA. A course of chronic intermittent treatment of a CF patient with a first and second compound of the present invention may be modified at the discretion of the attending physician in accordance with routine medical practice.

There is also provided in the invention a composition for the treatment of cystic fibrosis in a mammal, preferably a human patient. The composition comprises a first compound in a therapeutically effective amount to enhance the trafficking of a mutant CFTR polypeptide to the surface of an epithelial cell in a patient, and a second compound in a therapeutically effective amount to increase the chloride ion transport activity of a mutant CFTR polypeptide at the surface of an epithelial cell. The first and second compounds, as well as the therapeutically effective amounts thereof, are the same as those described hereinabove. In a particularly preferred aspect, the composition is a pharmaceutical composition contained within a biologically compatible buffer or a pharmaceutically acceptable carrier.

Preferably, the first compound is butyrate, phenylbutyrate, 4-phenylbutyrate, and/or a biologically active analog of butyrate or phenyl butyrate. The second compound may preferably be an isoflavone and/or a flavone. A preferred second compound is genistein, or a biologically active analog thereof.

In a preferred embodiment of the invention, the mutant CFTR polypeptide is ΔF508-CFTR. The invention, however, should not be construed to be limited solely to the treatment of CF patients having this mutant form of CFTR. Rather, the invention should be construed to include the treatment of CF patients having other mutant forms of CFTR with similar characteristics, including, but not limited to ΔI507, S549R, S549I, A559T and N1303K.

As used herein, the term “pharmaceutically-acceptable carrier” means a chemical composition with which an appropriate first compound and second compound may be combined and which, following the combination, can be used to administer the first and second compounds to a mammal.

The pharmaceutical compositions useful for practicing the invention may be administered to deliver a dose of between 1 nanogram per kilogram per day and 600 milligrams per kilogram per day. In one embodiment, a dose of the first compound is administered which results in a plasma concentration from about 50 micromolar to about 5 millimolar and a dose of the second compound is administered which results in a plasma concentration from about 10 micromolar to about 5 millimolar in the mammal. In a preferred embodiment, administration of a dose which results in a plasma concentration of the first compound of about 0.1 millimolar to about 2 millimolar in an affected epithelial cell of a mammal, and a concentration of the second compound of about 1 micromolar to about 100 micromolar in an affected epithelial cell of the mammal is performed.

Pharmaceutical compositions that are useful in the methods of the invention may be administered systemically in oral formulations, intravenously, parenterally, or topically in various formulations. In addition to one or more active ingredients, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer therapeutic agents according to the methods of the invention.

The invention encompasses the preparation and use of pharmaceutical compositions comprising one or more compounds useful for the treatment of CF as active ingredient(s). Such a pharmaceutical composition may consist of the active ingredient(s) alone, in a form suitable for administration to a subject, or the pharmaceutical composition may comprise the active ingredient(s) and one or more pharmaceutically acceptable carriers, one or more additional ingredients, or some combination of these. The active ingredient(s) may be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition and is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to other animals. Techniques to modify pharmaceutical compositions suitable for administration to humans to render the compositions suitable for administration to animals are well known, and can be performed by a skilled veterinary pharmacologist. Subjects to which administration of the pharmaceutical compositions of the invention is contemplated include, but are not limited to, humans.

Pharmaceutical compositions that are useful in the methods of the invention may be prepared, packaged, or sold in formulations suitable for oral, parenteral, intranasal, buccal, or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which generally comprises a carbon-containing liquid molecule and which exhibits a less polar character than water. A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these agents in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through, for example, a surgical incision. In particular, parenteral administration is contemplated to include, but is not limited to, subcutaneous, intraperitoneal, intramuscular,and kidney dialytic infusion techniques. The endoscopic procedure, endoscopic retrograde cholangiopancreatography (ERCP), can be utilized to intubate the pancreatic/biliary duct directly and thereby provide means to deliver drugs directly to the pancreas.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (wlw) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, contain 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating,agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

The pharmaceutical composition may be administered to a mammal as frequently as several times daily, or it may be administered less frequently, such as once a day, once a week, once every two weeks, once a month, or even less frequently, such as once every several months or even once a year or less. In another aspect, the pharmaceutical composition may be administered to a mammal following a chronic intermittent treatment schedule. The frequency of the dose will be readily apparent to the skilled artisan and will depend upon any number of factors, such as, but not limited to, the type and severity of the CF being treated, the type and age of the animal, etc.

Also included in the invention is a kit for treating cystic fibrosis in a mammal, preferably a human patient. The kit comprises an instructional material which directs the use of the components of the kit for performing the function of treating cystic fibrosis in a patient. The kit also comprises a first compound in a therapeutically effective amount to enhance the trafficking of a mutant CFTR polypeptide to the surface of an epithelial cell in the patient. The kit further comprises a second compound in a therapeutically effective amount to increase the chloride ion transport activity of the mutant CFTR polypeptide at the surface of the epithelial cell.

In one embodiment, the kit further comprises a device suitable for providing delivery in an aerosolized formulation of one or more of the first compound and the second compound. The device may be any device known in the art or described herein for providing delivery or administration of a compound in an aerosolized formulation to a patient.

The invention is now described with reference to the following Examples. The Examples are provided for the purpose of illustration only and the invention should in no way be construed as being limited to the following Examples, but rather should be construed to encompass any and all variations which become evident as a result of the teaching provided herein.

### EXAMPLE I

### A combination therapy for the treatment of CF

This Example provides a protocol for topical administration of the compound genistein following treatment with sodium 4-phenylbutyrate to augment the in vivo chloride ion transport function of ΔF508-CFTR in patients afflicted with CF. This in vivo chloride ion transport activity can be assessed using Nasal Potential Difference Measurements in ΔF508-Homozygous CF patients.

### Materials and Methods

### Patient Selection

Patients are eligible for entry in the study if they are ≧18 years of age, have CF, are homozygous for the ΔF508-CFTR mutation, are medically stable and are able to give informed consent.

There are no exclusions for gender or race in the study, although the prevalence of CF in the Caucasian population makes it likely that the vast majority of study patients are Caucasian. Exclusion criteria include any one or more of the following: pulmonary exacerbation of CF within the last month; cancers requiring treatment in the last five years (except those that have been cured, or which carry a good prognosis, such as non-melanoma skin cancer, cervical cancer in situ); GI disease (history of hepatitis or inflammatory bowel disease, or liver function test's (LFT's) >3-fold above upper limit of normal at screening visit); concurrent participation in another phase I trial; pregnancy or being less than 3 months post partum; breast feeding or being within 6 months of having completed breast feeding; unwillingness to undergo pregnancy testing or to use appropriate contraception during the study; psychiatric disorder which would impede study conduct; uncontrolled diabetes; and medication use or other conditions that may serve as exclusion criteria. Only adults are eligible for study entry.

A total of 24 patients will be enrolled in the study, 12 patients in the 20 grams/day group and 12 in the 30 grams/day group. Each dosage group will include 4 patients who receive placebo, and 8 which receive 4PBA. The unequal distribution of placebo and 4PBA subjects facilitates recruitment of subjects. The favorable results of the Pilot 4PBA trial (Rubenstein and Zeitlin, 1998, Am. J. Resp. Crit. Care Med. 157: 484-490) are well known in the CF community, and patients are typically more willing to enter the trial if there is a greater chance that they will receive the active agent than placebo. The details of the power calculation which leads to these required enrollments are described below.

The ethnic profile of CF patients indicates that more Caucasians are recruited than any other race. The ΔF508-CFTR mutation is 6 times more common in Caucasians than Asians or African-Americans. Toxicity is not anticipated in pregnancy with 4PBA or genistein. As a precaution, however, participants were asked to either abstain from sexual intercourse or to use an approved method of contraception for the duration of the study.

An overview of the study timetable is shown in Table 2 and described below in detail:

TABLE 2

| | −7 | 0 | | | | |
|---|---|---|---|---|---|---|
| Day | (screening) | (entry) | 4 | 7 | 14 | 21 |
| History | X | x | x | x | X | x |
| PE | X | x | x | x | X | x |
| Nasal PD | | x | x | x | X | x |
| Spirometry | X | x | x | x | X | x |
| Phlebotomy | X | x | x | x | X | x |
| Urine | X | | | | | |
| Pregnancy | | | | | | |
| Test | | | | | | |
| (Females) | | | | | | |

← Study Drug Treatment →+TZ,1/32

The overall duration of the study planned for each patient is 4 weeks. One patient has completed the course of the double-blind study described below. The study was conducted entirely on an outpatient basis and required a total of six outpatient visits of approximately two hours each. These visits entailed clinical evaluations, including history and physical (including mental status) examination, spirometry to monitor pulmonary function, and phlebotomy for routine metabolic (comprehensive metabolic panel, alanine amino transferase (ALT), gamma glutamyl transferase (GGT), and Uric Acid) and hematological (i.e., complete blood count, prothrombin time/ partial thromboplastin time) laboratory parameters. Specialized techniques for the determination of physiologic measures of CFTR function in vivo (Nasal Potential Difference) were performed at each visit after the screening evaluation and are described below.

After identification of eligible subjects by CFTR genotype on chart review and after informed consent was obtained, patients underwent a complete history, physical exam, and spirometry during the first visit (day 0). Monitoring chemistries and hematologic parameters were obtained using standard venipuncture techniques. A total of 7-10 milliliters of blood was required for these studies. Serum samples were obtained for measurement of genistein levels both before and after nasal potential difference (NPD) measurements on the first visit, as subjects who consumed a diet high in soy products may have measurable background genistein levels which can distort the outcome of the study if not quantitated and considered in the data analysis.

NPD measurements are a physiologic measure of in vivo CFTR function which were to be performed at each visit. The basic protocol for NPD was performed as follows. Baseline transepithelial potential difference across the nasal epithelia was measured by perfusing under the inferior nasal turbinate using sterile Ringer's solution through a PE-50 soft catheter probing “electrode.” The potential was measured against a subcutaneous reference “electrode” bridge created by inserting a 25 gauge butterfly needle filled with Ringer's solution just under the skin of the forearm into the extracellular fluid space. Aseptic technique was used for insertion of this electrode, and, if desired, the site of electrode insertion was topically anesthetized with a eutectic mixture of lidocaine/prilocaine (EMLA) cream. The electrode bridges were linked Ag/AgCl reference cells connected to a high impedance voltmeter. A stable baseline was established by pumping a superfusion of Ringer's solution at a flow rate of 2 milliliters per minute to facilitate mapping of the inferior turbinate in 0.5 centimeter increments to locate the point of maximal potential difference. This point was relocated and the solution was changed to 0.1 millimolar amiloride in Ringer's solution administered at 5 milliliters per minute for 2 minutes. The potential difference was continuously recorded and typically depolarized with the perfusion of amiloride. This was reflective of inhibition of epithelial cell sodium transport. In order to allow observation of epithelial cell chloride transport, the perfusion solution was changed to a chloride-free Ringer's solution (with gluconate substituted as the counterion for chloride) still containing amiloride at 2 milliliters per minute for 2 minutes. Finally, the solution was switched to 0.1 millimolar isoproterenol in low chloride/amiloride at 5 milliliters per minute for 3 minutes to stimulate cAMP accumulation and activate the CFTR. Measurements were repeated in the contralateral nostril and data averaged prior to analysis.

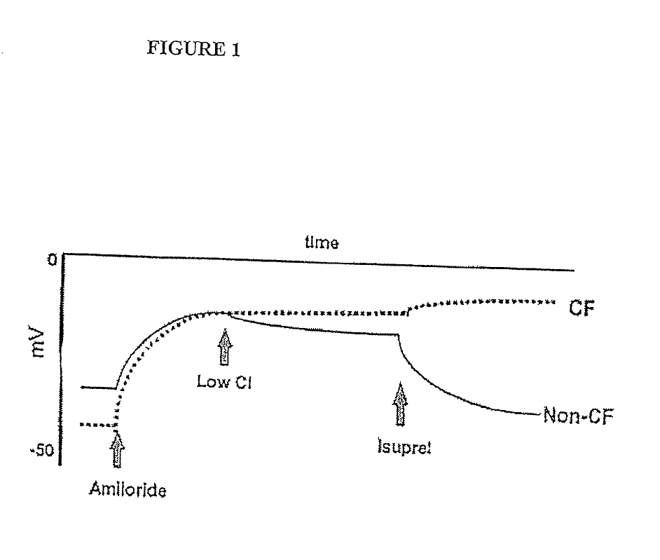

During these perfusions, the subject was positioned such that the perfusate dripped from the nose. This arrangement further minimized the potential for systemic absorption of these agents, which were present at such low concentrations that systemic side effects were unlikely. In typical NPD measurements, a CF pattern was represented by a baseline of less than or equal to 30 mV (−30 to −75 mV), a large depolarization following amiloride treatment, and no sustained hyperpolarization following low chloride/amiloride or isoproterenol/low chloride/amiloride treatment. The wild type response was a baseline of greater than or equal to −30 mV (−5 to −30 mV), a smaller depolarization following amiloride treatment, and a 10 mV or greater hyperpolarization following isoproterenol/low chloride/amiloride treatment. The best discriminator between a CF and non-CF pattern was isoproterenol/low chloride/amiloride response, wherein a 10 mV or greater hyperpolarization was inconsistent with a diagnosis of CF. FIG. 1 depicts typical results of NPD measurements in patients with CF and in non-CF patients.

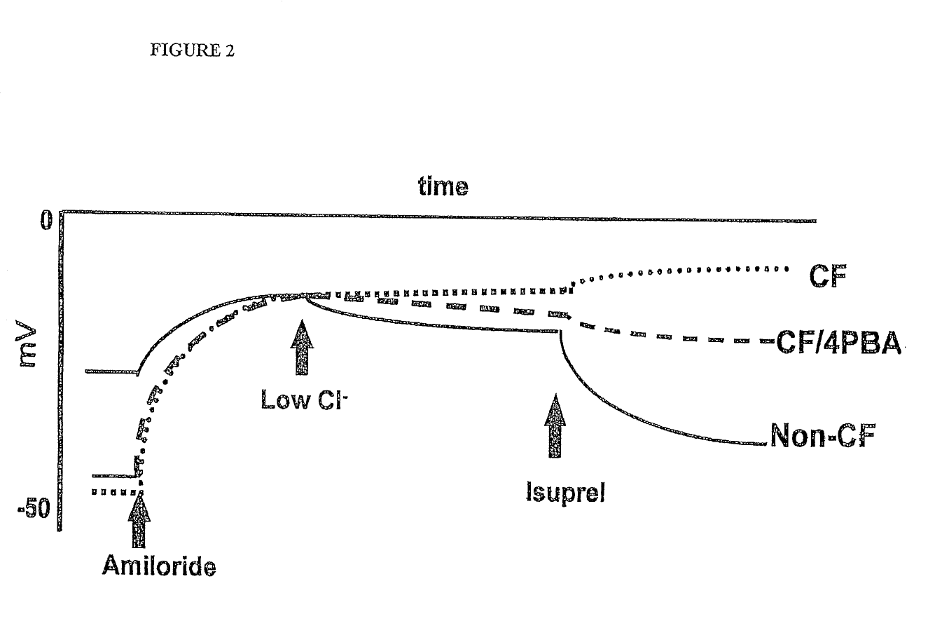

FIG. 2 depicts typical results of nasal potential difference measurements in patients homozygous for the ΔF508-CFTR mutation after therapy with 4-phenyl butyrate (4PBA)

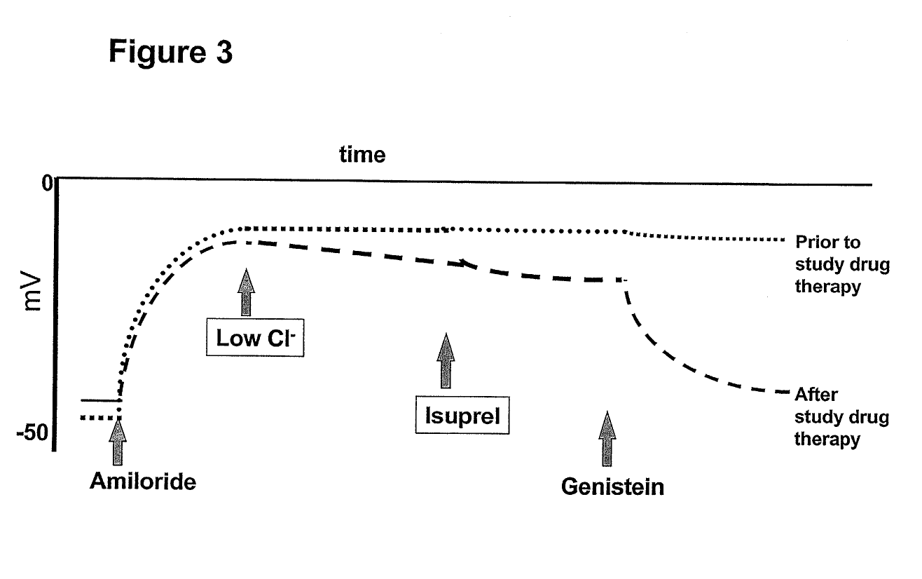

FIG. 3 depicts results of nasal potential difference measurements for a CF patient who was homozygous for the ΔF508-CFTR mutation and was treated in a blinded clinical trial to determine the combined effects of administration of 4PBA and genistein. The patient demonstrated a response to blinded study drug therapy that was consistent with that observed in patients who had received 4PBA in previous clinical trials (depicted in FIG. 2). The patient had a ⅔ chance of receiving 4PBA and a 100% chance of receiving genistein in this study.

The NPD procedure included, on days 1 and 7, after the isoproterenol/low chloride/amiloride perfusion, a perfusion of 50 micromolar genistein in low-chloride Ringer's solution in the continued presence of amiloride and isoproterenol for 3 minutes at 5 milliliters per minute. This technique for assessing genistein efficacy in vivo is similar to that recently reported for the studies of CF subjects with G551D mutations (Illek et al., 1999, Am. J. Physiol. 277: C833-C839). The total exposure to genistein at each of five treatments was about 0.4 milligrams, greater than 80% of which was present in the perfusate which drained from the nose to be collected. The total exposure of patients to 2 milligrams of genistein was far lower than the 16 milligram per kilogram oral dose used in Phase I trials of PTI G-2535 and G-4660. PTI G-2535 and G-4660 are commercially available formulations prepared according to current good manufacturing procedures (cGMPs) which contain a mixture of isoflavones, and are predominantly composed of genistein (Protein Technology Incorporated, St. Louis, Mo.).

After the initial evaluation and baseline measurements, subjects received a randomized, double-blinded study agent (4PBA or placebo) to take for one week on a three times a day (t.i.d.) schedule. 4PBA is currently available in 500-milligram tablets only. A placebo of sodium gluconate, which was identical in appearance to the 4PBA tablet, was administered to control subjects. Patients were instructed to take 13, 13, and 14 tablets on a t.i.d. schedule (for the 20 grams per day group) or 20 tablets 3 times for the 30 grams per day group. Subjects were asked to keep a symptom diary, as well as a diary of missed doses and circumstances surrounding such events.

Patients returned in the midst of the study (day 4) and at the end of the study drug treatment period (day 7) for repeated complete evaluations. Comprehensive evaluations were also performed weekly during a 2 week washout period. The total amount of blood drawn during the protocol was estimated to be 50 milliliters.

### Randomization

This study was performed in a randomized, placebo-controlled, double-blind fashion. In view of the positive results in previous 4PBA clinical trials (Rubenstein and Zeitlin, 1998, Am. J. Respir. Crit. Care Med. 157: 484-490), patients were randomized with a ⅔ probability of receiving 4PBA and a ⅓ probability of receiving placebo. Patients had a 100% probability of receiving genistein. A pharmacy department performed the randomization and delivered the coded pharmaceutical agent to the investigators. The pharmacy retained the coding sheet. The code was not broken until an entire group at a given dosage had completed the study, or unless an adverse event or toxicity occurred wherein it was essential that the agent assignment be known in order to administer appropriate medical care.

### Discontinuation

The study protocol could be discontinued at any time at the request of the patient. Additionally, it could be discontinued if any significant adverse event criteria were met. Throughout the study, the patients had access to the investigator directly by beeper and were instructed to contact the investigators for any medical concerns.

Primary safety and toxicity criteria included mental status, sleepiness score, electrolytes, chemistries, blood count, weight, pulmonary function, and gastrointestinal distress. Secondary outcomes were a change in taste in the mouth (a side effect peculiar to 4PBA), change in body odor, mild abdominal discomfort, headache, small decrease (less than or equal to 15% decrease) in pulmonary function [forced expiratory volume in one second (FEV1) or forced vital capacity (FVC)], and sore throat. In general, study toxicity criteria conformed to Common Toxicity Criteria of the National Cancer Institute, with CF-specific toxicity graded according to standards developed in conjunction with the CF Foundation Therapeutic Development Network. There were also a number of study-specific toxicities. The mental status assessment was performed using the Folstein Mini Mental Status Exam at each visit. A score of 24 or more out of 30 was considered normal and a score of ≦16 was indicative of significant toxicity.

Other significant toxicities were defined as follows:

a) Serum sodium, <125 or >150 meq/l; b) Liver functions, >3-fold rise from baseline; c) Platelets, <50,000; d) White blood count, >3-fold rise from baseline; e) Spirometry, any >30% decrease in FEV1 or FVC from baseline that persisted after administration of a bronchodilator [e.g., 2.5 mg of albuterol by nebulizer or 2 puffs of an metered dose inhaler (MDI) with spacer device] or f) Distal ileal obstruction syndrome requiring therapy with enemas or oral cathartic agents.

If a significant adverse event as described above had occurred, the study drug would have been suspended and the patient monitored through the washout period. If more than 2 patients in the 20 gram dose group who, after unblinding, had received 4PBA and had significant adverse events, the study would not have been permitted to progress to the 30 gram dose group. Six of the patients in the study have received 30 grams per day and this dosage group has since been discontinued. Secondary outcome events as described above were treated symptomatically and did not dictate a change in protocol.

### Study Drugs

4PBA (trade name Buphenyl) is manufactured by Medicis, Inc., in Phoenix, Ariz.. The FDA approved application for 4PBA as a chronic use therapy agent for patients with defects in the urea cycle which lead to hyperammonemia. In this use, 4PBA acts as a pro-drug for phenylacetate, which is formed from 4PBA by beta-oxidation. Phenylacetate acts as a sink for waste ammonia by its conjugation with glutamine to form phenacetylglutamine which, in turn, is excreted in the urine. The standard adult dose treatment of 4PBA is 20 grams per day given orally in three divided doses. The standard dosage and route of delivery were used throughout the study. 4PBA, in its FDA approved use as a chronic therapy for subjects with urea cycle disorders, has proven to be a very safe agent. The only commonly reported side effects of 4PBA are mild stomach upset and an occasional bad taste in the mouth. Significant severe or irreversible side effects have not been reported.

Genistein for oral use is manufactured by Protein Technology Inc., (St. Louis, Mo.) and has been used in Phase I clinical trials as a chemopreventative agent for prostate and breast cancers. In these trials, there were no side effects noted at a dose of 16 milligrams per kilogram per day. There is currently a Phase II trial of genistein approved for chemoprevention of prostate cancer and an application pending for a Phase II trial for chemoprevention of breast cancer.

### Results

The primary physiologic outcome measure of CFTR function was the change in nasal potential difference (NPD). In a previous report, (Rubenstein and Zeitlin, 1998, Am. J. Respir. Crit. Care Med. 157:484-490), the standard deviation of the NPD response to Isuprel and Low chloride perfusion (the most sensitive index of CFTR function) was ±2 mV. Assuming that a significant improvement in NPD from baseline is 5 mV (the difference between a CF and a non-CF response is greater than 10 mV), then 5 patients in each treatment group at each dose level yielded about 90% power to detect a difference between groups at a significance of 0.05. The same power of study can be accomplished using a total of 12 patients in each group with 8 receiving 4PBA and 4 receiving placebo.

The efficacy of the genistein perfusion was also a primary concern. For patients with the G551D mutation reported in the literature, there was an average of 2.4 mV repolarization of the NPD during genistein perfusion with a standard deviation of 1.2 mV. It was assumed, based on in vitro data (Illek et al., 1999, Am. J. Physiol. 277:C833-C839), that ΔF508-CFTR-homozygous patients who received 4PBA would respond to genistein perfusion with similar repolarizations as those reported for the G551D patients, while the ΔF508-CFTR-homozygous patients who received placebo would not respond to genistein perfusion. If this assumption was correct, then this study including 8 subjects receiving 4PBA and 4 subjects receiving placebo had greater than 95% power to detect a significant difference at a significance of 0.05.

### EXAMPLE II

### Genistein restores functional interactions between ΔF508-CFTR and EnaC in Xenopus oocytes

The cystic fibrosis transmembrane conductance regulator (CFTR), in addition to its Cl⁻channel properties, is involved in regulatory interactions with other epithelial ion channels including the Epithelial Sodium Channel, EnaC. The open probability (Pₒ) of wild type CFTR Cl⁻channels is increased significantly when CFTR is co-expressed in Xenopus oocytes with αβγENaC and conversely, the activity of ENaC is inhibited after wild type CFTR activation. Notably, in cystic fibrosis airway epithelia, where CFTR activity is decreased, ENaC is hyperactive as indicated by a greater change in nasal potential upon perfusion of amiloride (see FIG. 1). The most common CFTR mutation, deletion of a phenylalanine residue at position 508 (ΔF508-CFTR), is defective both in protein trafficking to the apical plasma membrane and in Cl- conductance due to a reduced Pₒ. While the ΔF508-CFTR trafficking defect can be repaired by reduced temperature or pharmacologic agents such as sodium 4-phenylbutyrate (4PBA) or glycerol, it is not known whether the repaired ΔF508-CFTR retains the ability to regulate other ion channels such as ENaC, or whether ENaC can regulate ΔF508-CFTR. The following example investigates the regulatory interactions of ΔF508-CFTR and ENaC when expressed in the model system of Xenopus oocytes.

### Materials and Methods

### Expression of human CFTR (WT and ΔF508) and mouse ENaC in Xenopus oocytes

Human WT-CFTR, human ΔF508-CFTR, and mouse α−, β−and γ ENaC cRNAs were prepared using a cRNA synthesis kit (m-MESSAGE mMACHINE, Ambion Inc, Austin, Tex.) according to the manufacturer's protocol. cRNA concentrations were determined spectroscopically. Oocytes obtained from adult female Xenopus laevis (NASCO Fort Atkinson, Wis.) were defolliculated and maintained at 18° C. in modified Barth's saline (88 mM NaCl, 1 mM KCl, 2.4 mM NaHCO_{3,} 15 mM Hepes pH 7.6, 0.3 mM Ca(NO₃)_{2,} 0.41 mM CaCl_{2,} 0.82 MM MgSO_{4,} 10 μg/ml sodium penicillin, 10 μg/ml streptomycin sulfate, 100 μg/ml gentamicin sulfate). Each batch of oocytes obtained from an individual frog was injected with either α−, β−, and γ subunits of ENaC (0.33 ng/subunit), WT-CFTR (10 ng), ΔF508-CFTR (10 ng), or a combination of ENaC and CFTR (WT or ΔF508) cRNAs dissolved in RNase-free water using a Nanoject II microinjector (Drummond Scientific).

### Electrophysiological analysis

Whole-cell current measurements were made 24 to 48 hours after injection using the two-electrode voltage clamp method (GeneClamp 500 amplifier-Axon Instruments, Foster City, Calif.). Single oocytes were placed in a 1 ml chamber containing modified ND96 (96 mM NaCl, 1 mM KCl, 0.2 mM CaCl_{2,}5.8 mM MgCl_{2,}10 mM Hepes, pH 7.4), and impaled with micropipettes of 0.5-5 MΩ resistance filled with 3M KCl. The whole-cell currents were measured by voltage clamping the oocytes in 20 mV steps between −140 mV to +60 mV adjusted for baseline transmembrane potential. Whole cell currents (I) were digitized at 200 Hz during the voltage steps, recorded directly onto a hard disk and analyzed using pClamp 8 software (Axon Instruments, Foster City, Calif.). Ion replacement studies were performed in an identical manner except that N-methyl-D-glucamine (NMDG) replaced Na⁺in the ND96 solution.

The difference in whole-cell currents measured in the absence and presence of 10 μM amiloride was used to define the amiloride-sensitive Na⁺current that was mediated by ENaC. Activation of ΔF508-CFTR was accomplished by perfusion of the oocyte with buffers supplemented with 10 μM forskolin and 100 μM IBMX for 25 minutes. As indicated, this first step can be followed by an incubation with 10 μM forskolin, 100 μM IBMX and 50 μM genistein for 20 minutes. In all experiments, ΔF508-CFTR Cl⁻current was defined as the difference in the current measured prior to forskolin/IBMX stimulation and the current measured either 20 minutes after perfusion with forskolin/IBMX or 15 minutes after perfusion with forskolin/IBMX/genistein. Whole-cell currents were measured at −100 mV. All measurements were performed at room temperature.

All reagents used were purchased from Fisher Chemicals, except for forskolin, IBMX and genistein, which were purchased from Sigma Chemical Co.

### Statistics

Statistical comparisons were performed using the Student's t test. A pair wise t test was used for pre/post treatment in experiments using an individual oocyte. An unpaired t test was used to compare currents obtained from oocytes injected with a cRNA for a single transporter (i.e., ENaC or CFTR (WT or ΔF508)) versus oocytes co-injected with a cRNAs for both ENaC and CFTR (WT or ΔF508). P values <0.05 indicated a statistically significant differential.

### Results

### Expression of ΔF508-CFTR and ENaC in Xenopus oocytes

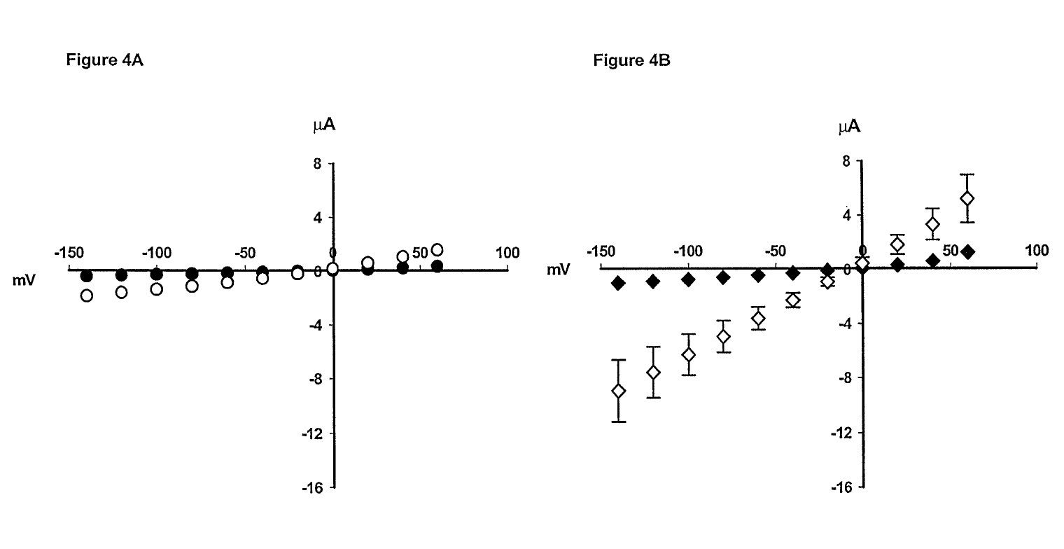

The Xenopus oocyte expression system was used to examine the functional expression of ΔF508-CFTR and its functional interaction with ENaC. The Xenopus oocyte is a model system which facilitates the expression and detection of functional CFTR with properties similar to those of endogenous CFTR (Bear et al., 1991, J. Biol. Chem. 266:19142-19145; Cunningham et al., 1992, Am. J. Physiol. 262:C783-C788; Drumm et al., 1991, Science 254:1797-1799) as well as functional ENaC (Li et al., 1995, Mol. Pharmacol. 47:1133-1140). Since oocytes are typically maintained at 18° C., this temperature enables the ΔF508-CFTR “trafficking defect” mutant to traffic to the membrane (Denning et al., 1992, Nature 358:761-764). Oocytes injected with 10 ng of human ΔF508-CFTR cRNA were bathed in a solution containing 10 μM forskolin/100 μM IBMX to activate endogenous protein kinase A and hCFTR. Whole-cell current was monitored by two-electrode voltage clamp (FIG. 4). In FIG. 4A, the I/V curves obtained in oocytes injected with ΔF508-CFTR cRNA before and after stimulation with 10 μM forskolin/100 μM IBMX were compared. The I/V curve remained linear. These results are characteristic of ΔF508-CFTR activity when expressed in Xenopus oocytes.

FIG. 4B shows the I/V curve of oocytes injected with rat αβγENaC (0.33ng/subunit) cRNAs. A large fraction of the current can be inhibited by 10 μM amiloride.

### Co-expression of WT-CFTR and ENaC in Xenopus oocytes

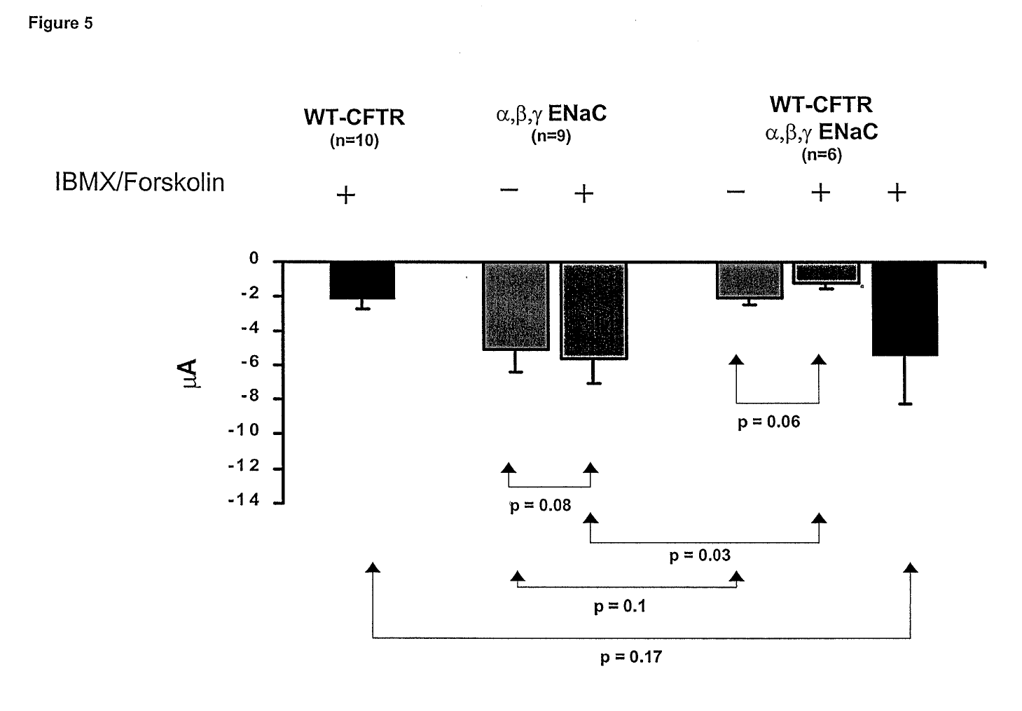

It has been reported that co-expression of WT-CFTR and ENaC in Xenopus oocytes inhibits ENaC-mediated current (Ji et al., 2000, J. Biol. Chem. 275:27947-27956; Mall et al., 1996, FEBS Lett. 381:47-52) and increases the cAMP-regulated conductance (Jiang et al. 2000, J. Biol. Chem. 275:13266-13274.). FIG. 5 shows the whole cell current measured at a holding potential of −100 mV in oocytes injected with human WT-CFTR (10 ng) or/and rat αβγENaC (0.33 ng/subunit). A 2.3±0.66 μA forskolin/IBMX stimulated current was measured in oocytes injected with WT-CFTR cRNA. In oocytes injected with αβγENaC, an amiloride-sensitive current was measured which was not altered in the presence of 10 μM forskolin/100 μM IBMX (5.1±1.4 μA versus 5.6±1.5 μA). These results indicated that ENaC is not sensitive to changes of intracellular cAMP, as shown previously (Mall et al., 1996, FEBS Lett. 381:47-52). In co-injected oocytes, an amiloride-sensitive whole cell current of 2.1±0.4 μA was observed in the absence of forskolin/IBMX. After CFTR activation by forskolin/IBMX, however, a decrease of the amiloride-sensitive whole cell current (1.2±0.29 μA) was observed. The forskolin/IBMX-stimulated whole cell current in oocytes co-injected with WT-CFTR and ENaC cRNAs (5.4±2.8 μA) was 2.4 fold larger than that obtained in oocytes injected with WT-cRNA alone (2.29+0.7 μA). These results confirm previous results and demonstrate that ENaC activates CFTR and CFTR inhibits EnaC.

### Co-expression of ΔF508-CFTR and ENaC

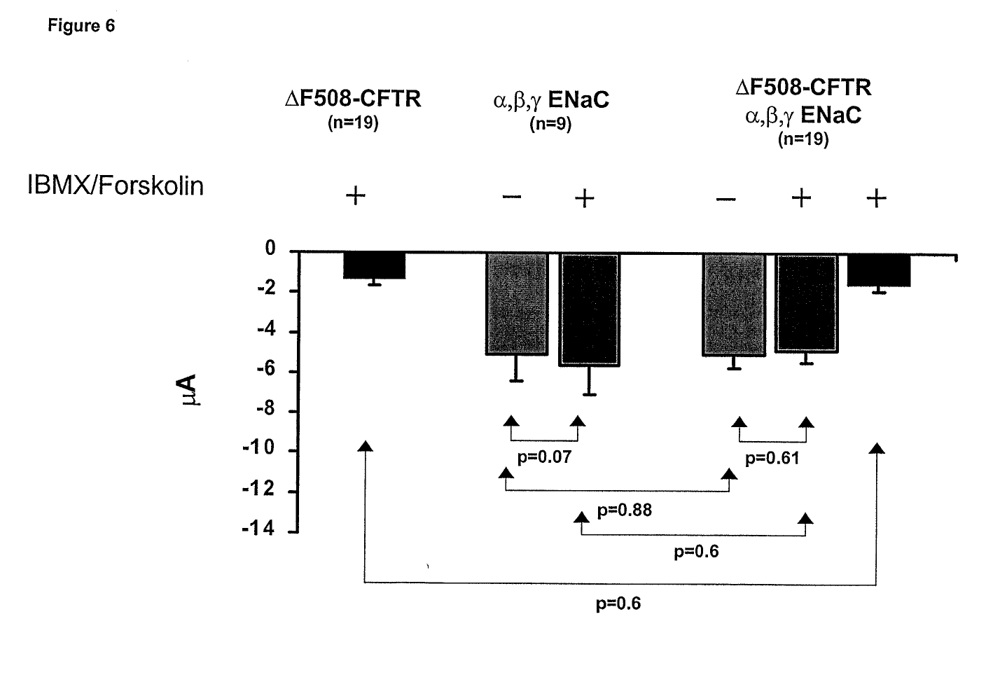

FIG. 6 shows that injection of ΔF508-CFTR cRNA into oocytes resulted in a 1.5±0.34 μA forskolin/IBMX-stimulated current and injection of αβγENaC cRNA resulted in 5.1±1.4 μA of amiloride-sensitive current which was not changed by addition of forskolin/IBMX (5.6 ±1.5 μA). ΔF508-CFTR and ENaC currents in ΔF508-CFTR/ENaC co-injected oocytes were similar to those observed in oocytes injected with ΔF508-CFTR or ENaC alone. These data were consistent with an absence of functional regulatory interactions between ΔF508-CFTR and ENaC in co-injected Xenopus oocytes (Mall et al., 1996, FEBS Lett. 381:47-52).

The ΔF508-CFTR forskolin/IBMX stimulated-current was less than that obtained with WT-CFTR (compare FIG. 6 to FIG. 5) which may reflect the lower open probability of ΔF508-CFTR relative to that of WT-CFTR. To explore further the regulatory interactions of ΔF508-CFTR and ENaC, the effect of genistein in the Xenopus system was investigated.

### Effect of genistein on regulatory interactions of EnaC with ΔF508 and Wild type CFTR

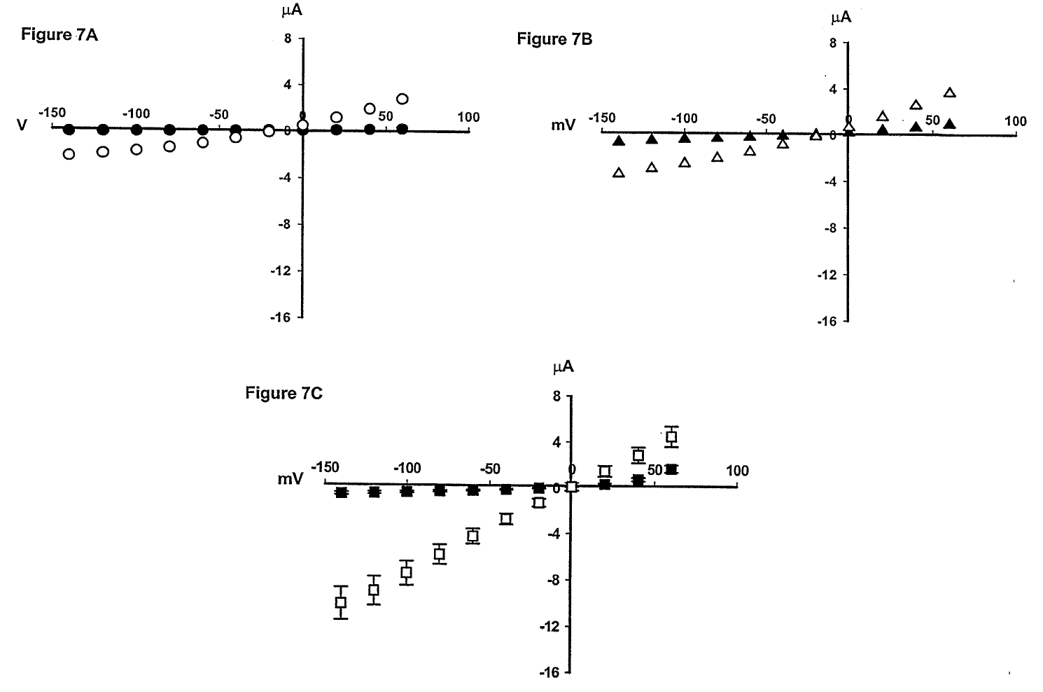

FIG. 7A shows the I/V curve of oocytes injected with human ΔF508-CFTR cRNA before and after stimulation with 10 μM forskolin, 100 μM IBMX, and 50 μM genistein. FIG. 7B shows the I/V curve of oocytes injected with human WT-CFTR cRNA before and after stimulation with 10 10 μM forskolin, 100 μM IBMX, and 50 μM genistein. FIG. 7C shows the I/V curve obtained in oocytes injected with rat αβγENaC (0.33 ng/subunit) cRNAs after stimulation with forskolin/IBMX/genistein and before and after addition of amiloride. A large fraction of the current can be inhibited by 10 μM amiloride. The amiloride sensitive current was slightly increased in the presence of 10 μM forskolin, 100 μM IBMX, 50 μM genistein (FIG. 7C; 7.09+1 μA versus 8.9±0.93 μA, p=0.004). In fact the amiloride-sensitive current was decreased in the presence of genistein alone, or when the genistein was added before the forskolin/IBMX (unpublished data).

### Effect of genistein in oocytes co-injected with ΔF508-CFTR and αβγENaC

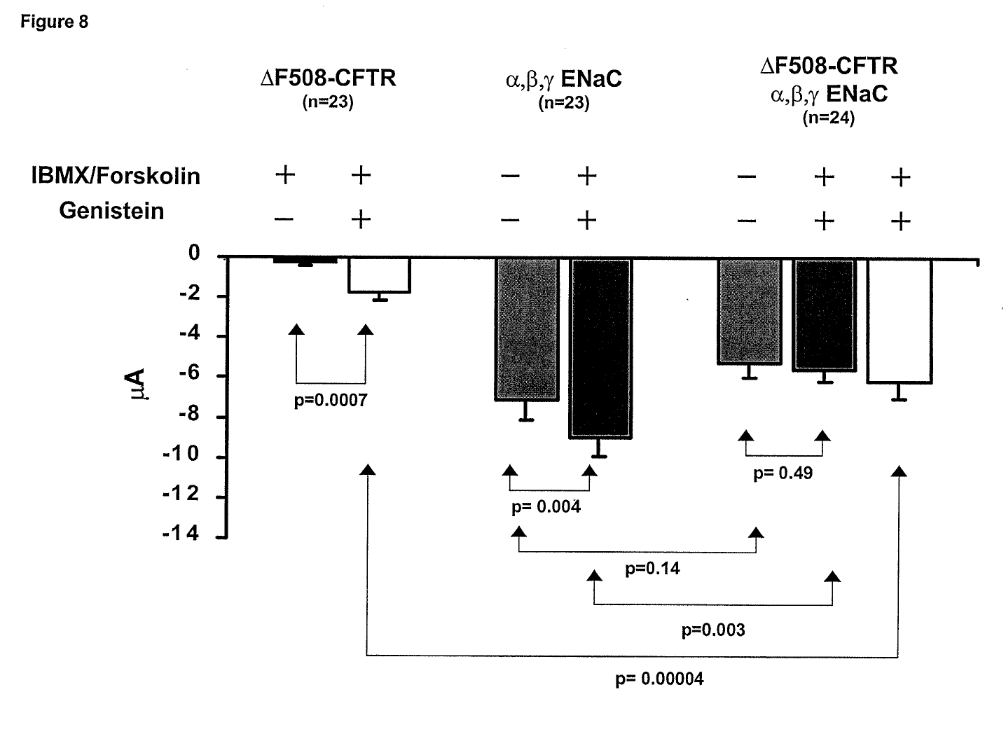

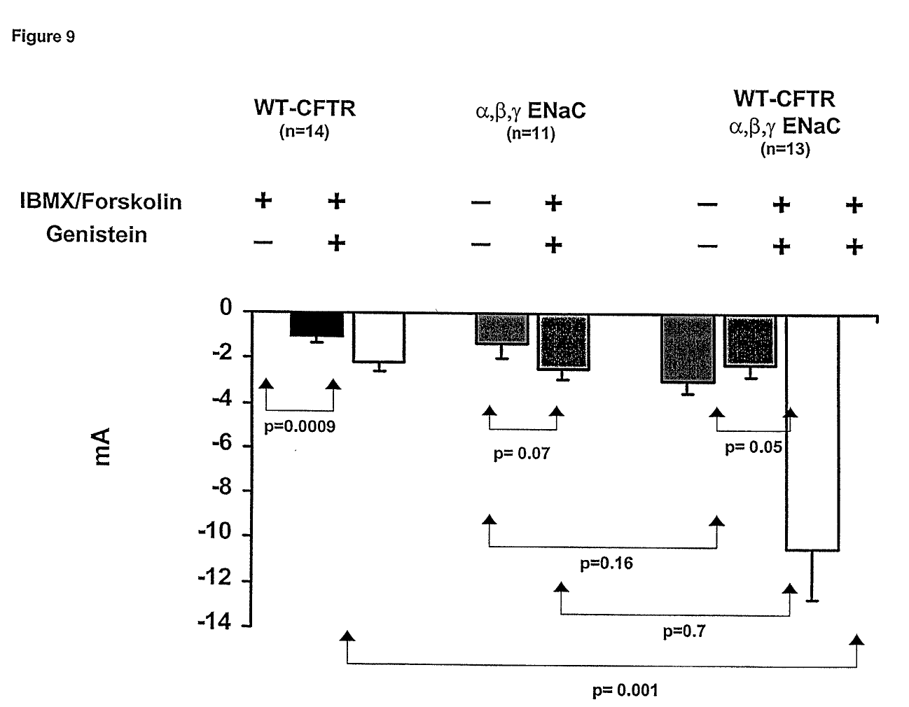

In oocytes injected with ΔF508-CFTR cRNA, the forskolin/IBMX-stimulated ΔF508-CFTR-mediated current was increased 5 fold by the addition of 50 μM genistein (FIG. 8; 1.82±0.4 μA versus 0.37±0.083 μA), whereas in oocytes injected with WT-CFTR cRNA, the forskolin/IBMX-stimulated current was increased 2 fold by the addition of genistein (FIG. 9; 2.57±0.45 μA versus 1.47±0.28 μA).

In oocytes co-injected with ΔF508-CFTR and αβγENaC cRNAs, the amiloride insensitive current stimulated by forskolin/IBMX/genistein was 3 times higher than that obtained in oocytes injected with ΔF508-CFTR alone (FIG. 8; 1.8±0.4 μA versus 6.2±0.87 μA, p=4.7×10⁻⁵). This suggested that ENaC was able to activate ΔF508-CFTR in the presence of forskolin/IBMX/genistein.

The amiloride-inhibited current obtained after stimulation by forskolin/IBMX/genistein was less than that obtained in oocytes injected with ENaC alone (FIG. 8; 8.9±0.9 μA versus 5.56±0.58 μA). This was consistent with further activation of ΔF508-CFTR by genistein, which restored wild type CFTR levels of inhibition of ENaC activity.

### Effect of NMDG on forskolin/IBMX/qenistein-stimulated whole-cell current

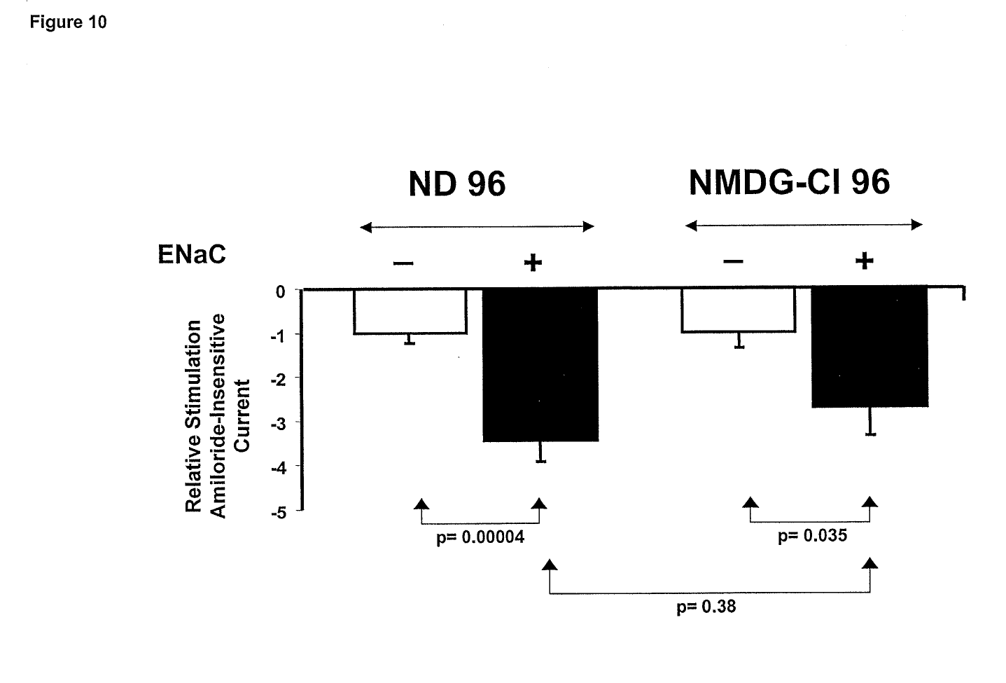

Replacement of Na+in the bath solution with the impermeant cation N-Methyl-D-Glucamine (NMDG) did not significantly affect the fold increase of forskolin/IBMX/genistein-stimulated current that accompanied ENaC co-injection (FIG. 10, 3.4+0.48 versus 3.35+1.23 fold increase of current). This suggested that the activation of ΔF508-CFTR by ENaC was not dependent on Na+transport by EnaC and the increase in amiloride-insensitive current, which was presumed to be ΔF508-CFTR mediated, was not due to decreased sensitivity of ENaC to blockade by amiloride.

### Conclusions

The results presented herein demonstrate that the lack of functional regulatory interactions between ΔF508-CFTR and ENaC in co-injected Xenopus oocytes after activation of ΔF508-CFTR by forskolin and IBMX can be restored by the addition of genistein. The data presented herein demonstrate that restoration of the functional inter-regulation that normally exists between wild type CFTR and EnaC is facilitated by genistein treatment ΔF508-CFTR and ENaC. In other words, ΔF508-CFTR and ENaC can function coordinately in the presence of genistein. The data, therefore, suggested that improving the ΔF508-CFTR trafficking defect alone was not sufficient to rescue a CF phenotype and demonstrated that a therapeutic regimen which combines rescue of both the ΔF508-CFTR trafficking defect (with 4PBA) and functional regulatory interactions (with genistein) will provide a more efficacious treatment for patients with CF.

The disclosures of each and every patent, patent application, and publication cited herein are hereby incorporated herein by reference in their entirety.

While this invention has been disclosed with reference to specific embodiments, it is apparent that other embodiments and variations of this invention may be devised by other skilled artisans without departing from the spirit and scope of the invention.

What is claimed is:

1. A method of treating cystic fibrosis in a mammal, said method comprising administering to said mammal a first compound in a therapeutically effective amount to enhance the trafficking of a mutant CFTR polypeptide to the surface of an epithelial cell in said mammal, said first compound being administered on a chronic intermittent schedule, thereby preventing induction of tolerance to said first compound, and administering to said mammal a second compound in a therapeutically effective amount to increase the chloride ion transport activity of said mutant CFTR polypeptide at the surface of said cell, wherein, the chloride ion transport function of said mutant CFTR polypeptide is enhanced at the surface of said cell, thereby treating said cystic fibrosis in said mammal.

2. The method of claim 1, wherein said mammal is a human.

3. The method of claim 1, wherein said mutant CFTR polypeptide is ΔF508-CFTR.

4. The method of claim 1, wherein said first compound is selected from the group consisting of butyrate, phenylbutyrate, 4-phenylbutyrate, and a biologically active analog of butyrate or phenyl butyrate.

5. The method of claim 11, wherein said second compound is selected from the group consisting of an isoflavone and a flavone.

6. The method of claim 1, wherein said second compound is genistein or a biologically active analog thereof.

7. The method of claim 1, wherein said first compound and said second compound are administered to said mammal together as components of the same composition.

8. The method of claim 1, wherein said first compound and said second compound are administered to said mammal as components of different compositions.

9. The method of claim 1, wherein said first compound is administered to said mammal prior to administering said second compound to said mammal.

10. The method of claim 1, wherein said first compound is administered to said mammal from about 4 hours to about 48 hours prior to administering said second compound to said mammal.

11. The method of claim 1, wherein said first compound is administered to said mammal orally.

12. The method of claim 1, wherein said second compound is administered to said mammal by a route selected from the group consisting of topically, orally, parenterally, by inhalation and intravenously.

13. The method of claim 11, wherein said epithelial cell is selected from the group consisting of a nasal epithelial cell, a lung epithelial cell, a pancreatic epithelial cell, an intestinal epithelial cell, a biliary epithelial cell and a sweat duct epithelial cell.

14. A kit for treating cystic fibrosis in a human patient, said kit comprising
a) a first compound in a therapeutically effective amount to enhance the trafficking of a mutant CFTR polypeptide to the surface of an epithelial cell in said human patient;
b) a second compound in a therapeutically effective amount to increase the chloride ion transport activity of said mutant CFTR polypeptide; and
c) an instructional material which directs the use of a) and b) for the function of treating cystic fibrosis in a human patient and optionally contains instructions for administration of the compounds on an intermittent treatment schedule.

15. The kit of claim 14, further comprising d) a device for providing delivery of one or more of said first compound and said second compound in an aerosolized formulation.

16. The method of claim 1, wherein said chronic intermittent treatment schedule comprises administration of said first compound for a duration of one to two weeks followed by a two to four week period wherein said first compound is not administered and said second compound is administered 2 days following the administration of compound 1.

17. The method of claim 1, wherein said chronic intermittent treatment schedule comprises administration of said first compound for a duration of three to four days followed by a two to four week period wherein said first compound is not administered.

18. The method of claim 1, wherein said chronic intermittent treatment schedule comprises administration of said first compound for a duration of three to four days followed by a two to four week period wherein said first compound is not administered.
